(19) United States
(12) Patent Application Publication
(10) Pub. No.: US 2010/0260861 A1
(43) Pub. Date: Oct. 14, 2010

(54) TREATMENTS WITH HELMINTHS

(76) Inventors: Joel Weinstock, Weston, MA (US); Vincent B. Young, Ann Arbor, MI (US)

Correspondence Address: FISH & RICHARDSON PC, P.O. BOX 1022, MINNEAPOLIS, MN, 55440-1022, US

(21) Appl. No.: 12/595,128

(22) PCT Filed: Apr. 10, 2008
(86) PCT No.: PCT/US08/59943

§ 371 Date: May 25, 2010

Related U.S. Application Data
(60) Provisional application No. 60/910,974, filed on Apr. 10, 2007.

(51) Int. Cl. A61K 35/56; C12Q 1/18; A61P 3/04

(52) U.S. Cl. 424/520; 435/32

(57) ABSTRACT

The invention relates to a method of treating obesity or IBS in an individual by administering a physiologically acceptable composition comprising a helminthic parasite, or an active variant thereof, that is of a type and present in an amount sufficient to treat the individual for obesity or IBS. The invention is not limited to obesity and IBS and is further drawn to conditions of health in which the microbiota profile for a population of microbes found within an individual contributes to a pathological condition.

This application claims the benefit of the priority date of U.S. provisional application No. 60/910,974, filed Apr. 10, 2007, the entire contents of which are hereby incorporated by reference in their entirety.

### TECHNICAL FIELD

This invention relates to compositions, including physiologically acceptable compositions, that include parasites and their use in the prevention and/or treatment of certain conditions. The condition can be a disease state associated with an undesirable profile of intestinal microbiota. More specifically, the condition can be obesity or irritable bowel syndrome (IBS).

### BACKGROUND

Helminths are parasitic animals (worms), which, depending on species, live in locations like the intestinal lumen, blood stream or muscles of a host organism. More than one-third of the world's population is colonized by helminthes, with the most common hosts being children living in warm climates and subject to poor sanitation. The infective forms of these organisms are spread through contact with contaminated soil, food or water. Before the 1940s, many children and adults in the United States carried helminths. Worm carriage was particularly common in rural areas of the South and in indigent populations of major cities (Elliott at al., FASEB J. 14(12):1848-1855, 2000). In the United States and Europe, helminthic colonization has steadily declined, but is still present; historically, for example, recent immigrants from countries described as less developed are often still affected (Salas at al., Arch. Intern. Med. 150(7):1514-1516, 1990) as are economically disadvantaged populations living in underserved regions of the United States (Healy et al., Public Health Rep 84(10):907-914, 1969). More generally, the prevalence of helminths is highest in warm climates and in populations subject to crowding, poor sanitation and impure food supply.

### SUMMARY

The present invention is based, in part, on the discovery that parasite preparations can alter the composition of a population of microbes in a host organism (e.g., in the host's gastrointestinal tract). We may refer to the composition of the microbial population, which can be complex, as the microbiota profile. The term “microbiota” refers to microorganisms, including bacteria, that populate a given environment (e.g., the gastrointestinal tract or a region therein). The microbiota profile can change when just a few types of microorganisms (e.g., one, two, or three types) become more or less prominent members of the larger population of which they are a part. For example, when a given type of bacterium, by genus or species, comes to constitute a larger or lesser proportion of a population of bacterium or a population of microorganisms of which it was previously a part, there is a change in the microbiota profile.

The present invention provides methods for treating an individual suffering from a condition that is characterized by and/or associated with a pathogenic microbiota profile in the gastrointestinal tract (e.g., in the mucosal epithelium of the gastrointestinal tract). The methods can be carried out by identifying an individual (e.g., a human patient) in need of treatment and administering a helminth parasite preparation that shifts the microbiota profile. The profile can be shifted, for example, toward one that is present or more commonly present in non-affected individuals. For example, the helminth parasite preparation may restore a non-pathogenic microbiota profile.

The present invention provides methods for treating an individual who is overweight, obese, or at risk of becoming obese. The methods can be carried out by administering to the individual a physiologically acceptable composition that includes one or more types of a helminthic parasite, or one or more active variants thereof. The parasite(s) will be of a type and present in an amount sufficient to treat the individual who is overweight, obese or at risk of becoming obese, as evidenced by weight loss over time. One may examine the microbiota profile before, during, or after treatment, and that profile may change as a result of treatment. The composition may be administered repeatedly for a short or extended period of time (i.e., the treatment may be chronic).

The present invention also provides methods for treating an individual wan has irritable bowel syndrome (IBS; for example, a patient who has received such a diagnosis). The methods can be carried out by administering to the individual a physiologically acceptable composition that includes one or more types of a helminthic parasite, or one or more active variants thereof. The parasite(s) will be of a type and present in an amount sufficient to treat the individual who has IBS, as evidenced by improvement in a sign or symptom of that condition. One may examine the microbiota profile before, during, or after treatment, and that profile may change as a result of treatment. The composition may be administered repeatedly for a short or extended period of time (i.e., the treatment may be chronic).

The helminthic parasite can be one that naturally colonizes humans but also colonizes animals (e.g., an animal other than a human, such as a mouse, rat, pig, dog, cat, marine mammal or bird). In addition, the helminthic parasite can be one that naturally colonizes animals (e.g., an animal other than a human, such as a mouse, rat, pig, dog, cat, marine mammal or bird) but also colonizes humans. Helminthic parasites in these categories include but arc not limited to parasites of the genus Ancylostoma, Anisakis, Ascaris, Gnathstoma, Hymenolepis, Pseudoterranova, Trichinella, Trichuris, and Toxocara. Further helminthic parasites in these categories include but are not limited to Ascaris suum, Hymenolepis nana, Trichinella spiralis, Trichuris vulpi, and Trichuris suis. Useful helminthic parasites that colonize both humans and birds include but are not limited to S. douthitti, Trichobilharzia ocellato, T. stagnicolae, T. physellae, and Gigantobilharzia huronensis.

Other useful parasites, which can be used alone or in combination with others described herein, include the helminthic parasites that naturally colonize humans. Helminthic parasites in this category include but are not limited to nematodes. Nematodes of the genus Ancylostroma, Ascaris, Enterobius, Hymenolepis, Necator, Nippostrongylus, Strongyloides, Trichinella, and Trichuris are in this category and include, but are not limited to, Ancylostoma duodenale, Ancylostoma brasiliense, Ascaris lumbricoides, Enterobius vermicularis, Necator americanus, Strongyloides steroralis, and Trichuris trichiura. This category of helminthic parasites further comprises platyhelminths, which may be trematodes or cestodes. Platyhelminths of the genus Fasciolopsis, Echinostoma, or Heterophyes are useful in the present compositions. Furthermore, helminthic parasites that reside in the biliary system such as Clonorchis sinensis, Opisthorchis viverrini, Opisthorchis felineus, Fasciola hepatica, or a parasite of the genus Schistosoma can be used. Suitable cestodes include those of the Diphyllobothrium species, Taenia saginata, Taenia solium, or Hymenolepsis nana.

The helminthic parasite can also be a filarial parasite or a lung fluke.

Other useful types of helminthic parasites are those that naturally colonize an animal other than a human (e.g., a mouse, rat, pig, dog, cat, marine mammal or bird). Helminthic parasites of the genus Angiostrongylus, Heligmosomoides, Nippostrongylus, and Trichuris, including but not limited to Heligmosomoides polygyrus, Nippostrongylus brasiliensis, or Trichuris muris are in this category.

The helminthic parasite can also be Schistosoma mansoni.

As noted above, the present compositions and methods can include active variants of helminthes. The active variant can he an extract of the helminthic parasite; an ovum or ovum extract of the helminthic parasite; an egg or egg extract of the helminthic parasite; a larvae or larval extract of the helminthic parasite; or a cercariae or cercariae extract of the helminthic parasite. The active variant may also be an isolated component of the helminthic parasite (e.g., a biochemically purified component or a fall-length or truncated expression product of helminthic parasite genomic DNA sequence obtained by recombinant techniques in non-native host cells). A given variant is said to be active where it performs, to any clinically beneficial extent, as the helminth from which it was derived or a preparation containing whole helminths of the same or varying types (e.g., of the same or a different species or genus than the helminth from which the variant was obtained). The helminths and/or active variants thereof can be used in combination. Moreover, the helminths and active variants thereof can be, or can be derived from, respectively, helminthes from any life stage (e.g., larval or adult).

As noted, the individual being treated can be a human, and any of the treatment methods described herein can include a step of identifying an individual in need of treatment.

The parasite-containing compositions can be formulated for oral administration and may include pharmaceutically acceptable materials used in preparing oral formulations, such as a filler, carrier, excipient, dispersant, or surfactant. Tablet formulations may further include an enteric coating. The composition may comprise about 50 to about 50,000 helminthic parasites, and administration may be repeated on a regular or irregular basis. For example, the composition may be administered (e.g., self administered) at least twice and on an approximately daily, weekly, biweekly, monthly, or bimonthly schedule.

Also provided are methods of identifying a helminthic parasite composition useful in treating an unwanted condition such as those described herein (e.g., obesity and/or IBS). These methods can include the steps of: (a) providing a helminthic parasite composition; (b) providing a population of microbes of determined microbiota profile; (c) exposing the population of microbes to the helminth parasite composition, and (d) determining the microbiota profile of the population of microbes. A helminth parasite composition that alters the microbiota profile of the population can be identified by comparing the microbiota profile before and a after exposure to the helminths. While any microorganisms can be assessed, this method may be used to identify a helminthic parasite composition that alters the microbiota profile to include a greater number or proportion of Bacteroidetes and a lesser number or proportion of Firmicutes. The population of microbes for which the microbiota profile is determined may exist in vivo (e.g., in an animal model or human patient) or ex vivo (as in a population of cultured cells). If desired, one can also determine the effect of the helminthic parasite composition on the immune system by, for example, examining the expression of a growth factor (e.g., a cytokine or interleukin).

The details of one or more embodiments of the invention are set forth in the accompanying drawings and the description below. Other features, objects, and advantages of the invention will be apparent from the description and drawings, and from the claims.

### BRIEF DESCRIPTION OF THE DRAWINGS

FIG. 1 is a series of bar graphs demonstrating that the immune response induced by concurrent infection with M. avium and S. mansoni is distinct from immune responses induced by either M. avium or S. mansoni alone. Concentrations of IFN-γ, IL-4 and IL-5 were measured in spleen cell supernatants of mice infected with M. avium, S. mansoni or both organisms. Splenocytes (4×10⁵/well)

FIG. 2 is a series of bar graphs demonstrating that the immune response induced by concurrent infection with M. avium and S. mansoni is distinct from immune responses induced by either M. avium or S. mansoni alone. Granuloma cells (4×10⁵/well) in 200 μL of medium were cultured in vitro at 37° C. for 48 hours in the presence or absence of the optimal concentration of PPD or SEA. Cytokines were quantified by ELISA.

FIG. 3 is a series of bar graphs showing the serum IgG1, IgE and IgG2a levels measured in mice infected with M. avium, S. mansoni or both (concurrent) according to one embodiment of the invention. Immunoglobulin concentration was determined by ELISA.

### DETAILED DESCRIPTION

Described herein are compositing that include one or more helminthes or active variants thereof that can be used to alters the composition of a population of microbes and/or to treat individuals who have, or who are at risk for attaining, excess weight or IBS.

### A. Helminthic Parasites

Use helminthic parasites include, but are not limited to two groups. The first group is helminthic parasites that naturally colonize humans, and the second group is helminthic parasites that colonize animals, but may, when administered to humans as a preparation, alter the structure and composition of a population of microbes in humans.

In the first group, helminthic parasites are elaborate multicellular worms with complex life cycles and development. The nematodes (non-segmented round worms), and the platyhelminths (flat worms) are two groups of helminths that colonize he human intestines. In accordance with the present invention, any one of a number of helminth parasites that naturally colonize humans or animals will provide the intended results.

Nematodes that frequently inhabit the human gut are Ascaris lumbricoides, Enterobius vermicularis (pin worm), Trichuris trichiura (whipworm), Ancylostoma duodenale and Necator americanus (hookworms), and Strongyloides stercoralis. Trichinella spiralis infests the small intestine briefly.

Platyhelminths include the trematodes cestodes. The most common adult trematodes that reside in the human intestines are Fasciolopsis, Echinostama and Heterophyes species. Those that live in the biliary system include Clonorchis sinensis, Opisthorchis viverrini and felineus, and Fasciola hepatica. Schistosoma dwell in the venous system, but several species chronically affect the gut by the passage of eggs through the intestinal wall. Adult cestodes commonly infecting humans are Diphyllobothrium species (fish tapeworm), Taenia saginata (beef tapeworm), Taenia solium (pork tapeworm) and Hymenolepsis nana (dwarf tapeworm).

Other helminths of interest include the filarial parasites and the lung flukes.

The second type of helminthic parasites that can be utilized include helminths that colonize animals. These include Trichuris muris (mouse whipworm), Trichinella spiralis, Nippostrongylus brasiliensis, Heligmonsomoides polygyrus and Hymenolepis nana, all of which are intestinal helminths that infect mice. Additionally, Angiostrongylus is a rat helminth. Trichuris suis and Ascaris suum are pig helminths that can infect humans. Trichuris vulpis, Toxocara species, Gnathostoma, and Ancylostoma are dog or cat helminths that also can infect humans. Anisakis and Pseudoterranova are nematodes of marine mammals that can transmit to humans. Bird schistosomes can transiently infect humans. Such schistosomes include S. douthitti, Trichobilharzia ocellata, T. stagnicolae, T. physellae, and Gigantobilharzia huronensis.

The helminthic preparation can include helminiths that naturally colonize humans and helminths that colonize animals but when administered to humans change the structure and/or composition of microbes in humans.

In some embodiments of the invention, the helminth parasite is a nematode and may be Ascaris lumbricoides, Enterobius vermicularis, Trichuris trichiura, Ancylostoma duodenale or Necator americanus, Strongyloides stercoralis or Trichinella spiralis.

The helminthic parasite can be a platyhelminth and may be a trematode or cestode, such as Fasciolopsis, Echinostoma or Heterophyes species, Clonorchis sinensis, Opisthorchis viverrini, Opisthorchis felineus, Fasciola hepatica, Schistosoma species, Diphyllobothrium species, Taenia saginata, Taenia solium or Hymenolepsis nana.

The helminthic parasite can also be a filarial parasite or lung fluke.

In preferred embodiments, the helminthic parasites are selected from the group consisting of Trichuris muris, Trichinella spiralis, Nippostrongylus prasiliensis, Heligmonsomoides polygyrus, Hymenolepsis nana, Angiostrongylus species, Trichuris suis, Ascaris suum, Trichuris vulpis, Toxocara species, Gnathostoma species, Ancylostoma species, Anisakis species and Pseudoterranova species.

It is preferred that the preparatory animals for parasite production and preparation are rasied in specific pathogen-free (SPF) (e.g., specific human pathogen free) environment.

### B. Helminthic Parasite Preparations

Viable Organism Preparations. Viable organisms can be administered in either egg, larval, cercarial, or encysted larval forms depending on the helminth. Helminths that can colonize humans and a preparatory animal may be utilized in the methods described herein.

The animal used to prepare the helminthic preparation may be manipulated to allow high (or improved) patency by the helminth. Such manipulation can include treatment with agents that are immunosuppressive (e.g., glucocorticoids or azathioprine); agents that impede Th2 effects (e.g., anti-histamines, anti-cytokines, or recombinant cytokines); or agents that influence intestinal motility (e.g., anti-cholinergics or opiates). The animal's diet can he altered to reduce coarse fiber content. The animal can be a rodent (e.g., a mouse, hamster, or rat), a farm animal such as a pig, a bird, or other preparatory animal.

Preparatory animals can be raised in specific pathogen-free (SPF) environments (e.g., environments free of specific human pathogens) according to methods known in the art. The animals can be tested to ensure an absence of human bacterial, mycobacterial, and viral pathogens. Methods for raising animals in SPF environment and advantages thereof are described in the art (see, e.g., Swindle, J. Invest. Surg. 9:267-271, 1995, which is hereby incorporated by reference). Swindle also lists several potential human viral and bacterial pathogens of concern. Accordingly, parasite preparations derived from animals raised in a specific human pathogen tree environment would not contain these human pathogens, and as such are distinct from parasite preparations prepared from animals raised in any SPF environment.

Eggs: Some intestinal helminths are acquired by ingestion of viable eggs. As noted above, helminths can be maintained in SPF preparatory animals. To harvest eggs, the animals are given a special diet low in coarse fiber. Animals are given an oral purgative to induce defecation. Stool is collected and enzymatically digested to free eggs, which can then be isolated from liquefied stool by, for example, flotation on density gradients, screen filtration, Visser filtration, or centrifugal elutriation. Preservation of eggs varies with the helminth used. Eggs from helminths that are resistant to dessication can be dried, compounded with inert material, incorporated into an coterie capsule, and refrigerated. Eggs from helminths that are susceptible to dessication can be preserved by refrigeration in liquid medium or by adding cryoprotectant and freezing in liquid nitrogen. Viable eggs are washed, mixed with chilled lactose-tree pudding or other vehicle at the location for delivery. Eggs stored in glycerol-based cryoprotectant may not require washing. Eggs from each lot can be tested for hatching rate to determine effective dosing. Eggs from each lot can also be tested to confirm the absence of bacterial and viral pathogens.

Larvae: Some helminths (i.e. hookworms) require a soil maturation phase before they can colonize humans. Eggs from these agents will be incubated under optimal conditions to mature the embryo, or hatch the egg and provide larval forms. Patients can be inoculated by subcutaneous injection or oral ingestion of viable larvae.

Cercariae: Some helminths have complex life cycles that utilize an intermediate host. The intermediate host sheds the form able to colonize humans. Cercariae are the form for trematode helminths (i.e. flukes) shed by intermediate hosts like snails. Cercariae are isolated from colonized snails grown in SPF conditions. Cercariae are washed. These may be preserved by adding cryoprotectant and freezing in liquid nitrogen. Thawed or fresh cercariae are washed and injected subcutaneously to inoculate patients. Samples from each lot are tested for the absence of pathogens and to determine an effective dose.

Encysted Larvae: Some helminths (e.g. tapeworms) form encysted larvae or cysticerci in intermediate hosts. It is the encysted larval form that initiates human colonization. Encysted larva are removed from intermediate hosts, for example, cattle or fish or plants grown in SPF conditions. Cysts are washed free of remaining host tissue. Cysts may be preserved by adding cryoprotectant and freezing in liquid nitrogen. Cysts are thawed or used fresh, washed, mixed with chilled lactose-free pudding or other vehicle at the location for delivery and fed to individuals. Samples from each lot are tested for absence of pathogens and to determine effective dose.

### B. Non-Viable Component Preparations

Non-viable components of helminthic parasites derived from eggs, larvae or adult worms may be used in the helminth parasite preparation.

Non-viable, intact schistosome eggs produce a strong Th2 response. Eggs are isolated front livers of preparatory animals (i.e. hamsters) grown under SPF conditions. Eggs are isolated by a method modified from that originally described by Coker and Lichtenberg (Proceedings of the Soc. For Exp. Biol. & Med. 92:780, 1956). The modifications consist of using phosphate buffered saline with glucose instead of 1.7% saline for incubation and washing steps along with decreasing the autolytic digestion time. These changes promote isolation or viable eggs. The method is as follows. Infect golden hamsters with 1000 to 1500 cercariae. Allow the infection to mature (6 to 7 weeks). Remove the livers from the animals and place in 600 mOsm sterile phosphate buffered saline containing 5% glucose, 100 U/ml penicillin and 100 mg/ml streptomycin. The livers are allowed to autodigest for 24 hours at room temperature. Pulse homogenize the livers at low speed for 3 minutes in a cold Waring blender. Incubate the homogenate with collagenase (2 mg/ml) and trypsin (2 mg/ml) at 32° C. for one hour. Filter the homogenate through 50 and 80-100 mesh sieves to remove clumps of tissue and debris. Recover the eggs from the filtrate by passing over a 325 mesh sieve. The eggs will not pass through the screen. Flush the eggs off of the screen and into a 50 ml polypropylene centrifuge tube. Wash the eggs by repeated low speed (400×g) centrifugation in sterile phosphate buffered saline with 5% glucose. The eggs must be free of any collagenous debris. Count an aliquot of eggs in a 1 ml Sedwick chamber to determine total egg number. Isolated eggs are suspended in saline and flash frozen in liquid nitrogen without cryoprotectant. This kills the egg. Thawed eggs are injected subcutaneously, intramuscularly or intravenously, or at sites of Th1 inflammation to elicit strong Th2 responses. Eggs from other helminths may also be utilized.

Component preparations may also be used that employ proteins, lipids, or carbohydrates isolated from parasite eggs. An example is schistosome soluble egg antigens (SEA). The method for preparing schistosome egg antigen has been previously described by Boros and Warren, J. Experimental Med. 132:488, 1970. The method is as follows. Washed eggs are resuspended at 50,000 eggs/ml of phosphate buffered saline, which is transferred to a glass tissue homogenizer. The eggs are homogenized on ice. To insure that all shells are broken and miracidia are disrupted, an aliquot (5 ml) is removed for microscopic inspection. Transfer the homogenate to ultracentrifuge tubes. Centrifuige at 100,000×g for 2 hours at 4° C. Recover the aqueous fraction (SEA) and determine the protein content. Store the SEA in small aliquots at −70° C. This method may require modification to isolate the parasite egg products that most strongly effect the structure and composition of microbes in a population, i.e., to achieve an optimal effective concentration, (100 μg SEA or 10,000 ova/animal). Eggs or egg components are tested to confirm the absence of pathogens and endotoxin.

Component preparations can also be developed from larvae and adult worms of helminthic parasites. Larvae or worms are isolated from preparatory animals grown in SPF conditions. Preparations that employ non-viable intact organisms or proteins, lipids, or carbohydrates isolated from the helminth are prepared and utilized in a manner similar to that previously described for helminth eggs.

Also encompassed in the present invention are preparations containing fractions or subfractions of the helminthic parasites, as well as preparations containing isolated proteins, polynucleotides, carbohydrates, lipids, etc. derived from helminthic parasites according to known methods in the art (e.g., as described in Williams et al., J. Infect. Dis. 170:946, 1994; Xu-Amano et al., J. Exp. Med. 178:1309, 1993; and Ausiello et al., J. Infect. Dis. 181:1989, 2000).

For example, fractions or subtractions may be prepared according to the method described in Thaumaturgo et al. (Mem. Inst. Oswaldo Cruz vol. 96 suppl. Vol. 96, Suppl.: 79-83, 2001), in this method, S. mansoni parasites were obtained by retrograde perfusion with heparinized saline (0.85% NaCl solution), 45 days post-infection (Pellegrino and Siqueira, Rev. Bras. Mal. D. Trop. 8:589-597, 1956), and used for preparing the different extracts.

Preparation of adult-worm-derived extract (SE)—Parasites were rinsed in PBS. Living worms were then allowed to stand at room temperature (28° C. to 30° C.) in fresh PBS (phosphate buffered saline) for 2-3 hours, and frozen at −20° C. After thawing, PBS suspensions of the worms (1 g worms/10 ml PBS) were filtered through a wire mesh, and centrifuged at 10,000 g for 1 hour at 4° C. (Tendler & Scapin, Mem Inst Oswaldo Cruz 76; 103-109, 1981; Tendler et al. Mem Inst Oswaldo Cruz 77: 275-283, 1982). The protein content of SE was assessed by Lowry's method (Lowry et al. 1951), and stock batches containing 1 μg/ml were stored at −20° C. Male and female extracts were prepared according to the same methodology, with parasites separated immediately after perfusion.

Preparation of a rapidly released “fraction” of adult-worm-derived extract (SEi): SEi preparation corresponded to the initial step of SE: after perfusion as described for SE, live worms were incubated for 2-3 hours at room temperature (28° C. to 30° C.), in PBS. Thereafter, adult worms were ^{.}filtered from incubation solution (SEi) in a wire mesh.

Preparation of SE2: Adult worms remained from SE initial preparation, were re-stored frozen (−20° C.) in PBS for one week (1 g worms/10 ml PBS). After thawing, PBS suspensions were filtered through a wire mesh, and centrifuged at 10,000 g for 1 h at 4° C.

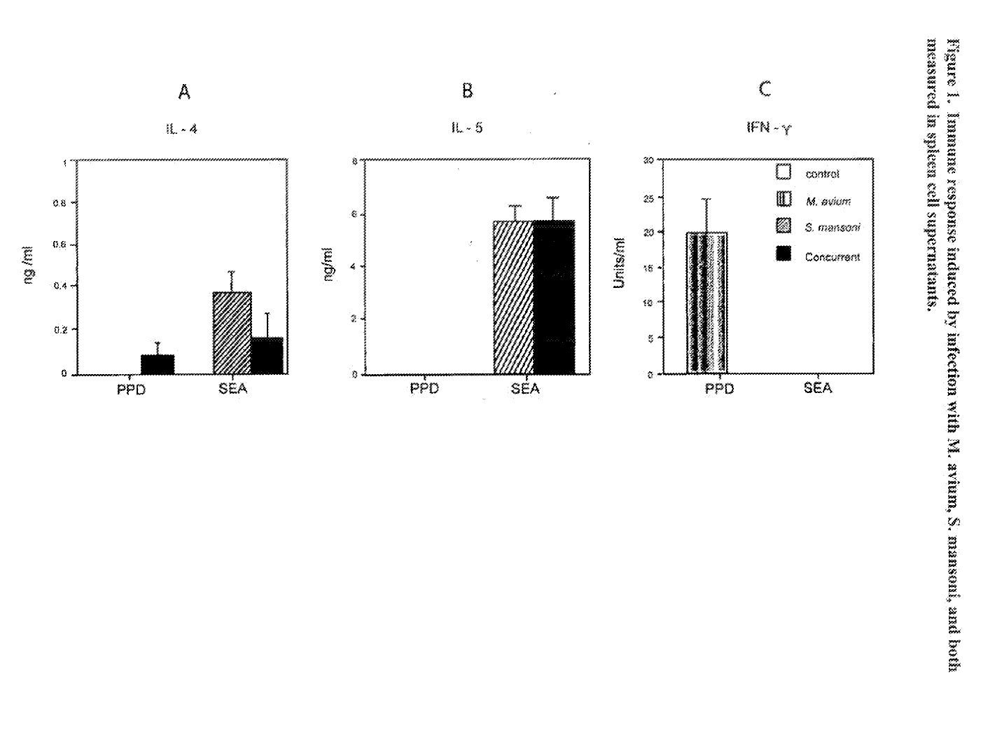

Parasite antigens can also be extracted as described in FIG. 1 of Thaumaturgo et al. (supra). Alternatively, parasite antigens may be prepared as described in Deelder et al. (Am. J. Trop. Med. Hyg. 29:401-410, 1980). For example, antigens may be prepared from SWAP prepared as soluble supernatant fluids from buffered saline homogenates of the respective life-cycle stage (Goes et al. Parasite Immunol. 11:695-711, 1989). SWAP was fractionated by anion-exchange chromatography on FPLC (last protein liquid chromatography), as previously described (Hirsch & Goes, Parasitology 112:529-535, 1996). Briefly, proteins were elated with 20 mM Tris-HCI, pH 9.6, in a multistep increasing gradient up to 1 M NaCl, interrupted by hold-gradient intervals at 0, 100, 280, 450, 600 and 750 mM. Flow-through fractions were concentrated by lyophilization. The concentrated material was dialyzed against 0.15 M phosphate-buffered saline (PBS), pH 7.4, sterilized by filtration and stored at −70° C. The protein content was measured according to Bradford microassay (Bradford, Analyt Biochem 72:248-254, 1976). Analysis of the six fractions, separated by 10% SDS-PAGE (sodium dodecylsulfate polyacrylamide gel electrophoresis) under reducing conditions (Laemmli, Nature 227;680-685, 1970), showed multiple protein bands, fraction III containing high (97 and 160 kDa), intermediate (52 and 56 kDa) and low (28 and 36 kDa) proteins. This fraction was called PIII and was used in different immunological assays.

Lipids may also be prepared as described in the art, for example, in van der Kleij et al. (Infection and Immunity 67: 5946-5950, 1999).

In one embodiment, lipids of S. mansoni adult worms (12 g [wet weight]) and eggs (1.6 g [wet weight]) can be extracted by the method described by Bligh and Dyer (Can. J. Biochem. Physiol. 37:911-917, 1959). The organic phase was dried by rotary evaporation, dissolved in 10 ml of chloroform, and applied to a 20-ml column of the anion exchanger TEAE-cellulose (Serva, Heidelberg, Germany) that was converted to the hydroxyl form. Lipids were eluted as described by Rouser et al. (Methods Enzymol. 14:272-317, 1969). According to this protocol, the fractions contain the following lipids: fraction 1, cholesterol, glycerides, and other neutral lipids; fraction 2, cerebrosides, glycerol diglycerides, phosphatidylcholine, and sphingomyelin; fraction 3, ceramidepolyhexosides; fraction 4, inorganic substances; fraction 5, phosphatidylethanolamine and free fatty acids; fraction 6, phosphatidylserine; fraction 7, none (washing step); and fraction 8, phosphatidic acid, cardiolipin, phosphatidylglycerol, phosphatidylinositol, and other acidic lipids. The presence of glycolipids in fractions 2 and 3 was confirmed by orcinol staining of the lipid fractions on HPTLC plates (J. Neurochem. 1:42-53, 1956).

Molecular biological techniques and recombinant methods well known in the art may be used to isolate, purify and produce Helminth components, which include but are not limited to nucleic acids and proteins derived from Helminths.

### C. Maintenance of Helminth Organisms

Helminths are cycled through intermediate and preparatory animals grown in SPF conditions. Samples of helminth populations are tested to ensure phenotypic stability such as colonization rates, fecundity, and susceptibility to anti-helminthics.

### D. Administration of Helminth Parasite Preparations and Pharmaceutical Formulations

Individuals may receive the infected form of the parasite (egg, cercariae or larvae) orally or parenterally depending upon the natural life cycle of the parasite selected. Alternatively, soluble worm or egg extracts can be given orally or parenterally.

With regard to intestinal and liver helminths and schistosomes, they begin producing ova that appear in the stool about 30-60 days after inoculation. Quantifying the eggs in the stool proves satisfactory for assessing adequacy and intensity of infection. Aliquots of stool are processed by sucrose floatation to determine the total number of eggs in each specimen. Flotation over sucrose solution is a method frequently used to isolate eggs from stool for accurate counting as reported by Koontz and Weinstock (Gastroenterology Clinics of N. America, 25:435, 1996).

The helminthic parasite compounds of the invention may be formulated for administration in any convenient way, and the invention therefore includes within its scope pharmaceutical compositions comprising the helminthic parasite compound in accordance with the invention adapted for use in humans. Such compositions may be presented for use in conventional manner with the aid of any necessary pharmaceutical carriers or excipients.

The helminthic parasite compound according to the invention may be formulated for injection, and therefore, vaccine use, and may be presented in unit dose form in ampules, or multidose containers, if necessary, with an added preservative. The compositions may also take such forms as suspensions, solutions or emulsions of oily or aqueous vehicles and may contain formulatory agents such as suspending, stabilizing, and/or dispersing agents. Alternatively, the helminthic parasite may be in powder form or reconstituted with a suitable vehicle, e.g. sterile-pyrogen-free water, before use.

If desired, such powder formulation may contain an appropriate non-toxic base in order to ensure that the powder is reconstituted with water, the pH of the resulting aqueous formulation being physiologically acceptable.

The helminthic parasite compounds may also be formulated as suppositories, e.g. containing conventional suppository bases such as cocoa butter or other glycerides.

The helminthic parasite compounds may also be formulated for oral dosage (as, for example, tablets or capsules) with conventional fillers, carriers, and excipients. The amount of parasite administered to the individual in need thereof is an amount sufficient to prevent or treat the condition, which may or may not encompass eradicating the symptoms. This amount may vary depending upon the condition being treated or prevented and the helminthic parasite, whether it is being administered intact, or as an egg, larvae, extract or cercariae.

Typically, when the parasites are administered for all conditions discussed herein, the amount ranges from about 50 to about 50,000. More particularly, this amount may range from about 500 to about 5,000. When eggs are utilized, about 500 to about 5000 may be utilized to treat the conditions disclosed herein. When extracts are administered, about 100 μg to about 10,000 μg are utilized to treat the conditions. When larvae and cercariae are administered, the dosages may range from about 500 to about 5,000 in each case.

For prevention or vaccine use, the amounts of the parasites may be 500-5,000.

Dosage of a parasite preparation may be monitored by determining the microbiota profile of a given tissue and by measuring Th1, Th2 or regulatory cell responses. Alternatively, the dosage may be monitored by evaluating the pathology or symptoms of a particular condition as known in the art or described herein.

### E. Determination of the Composition and Structure of a Population of Microbes

The microbiota profile may be determined for cell samples, tissue samples, organ samples, bodily fluids and fecal samples. The invention is not limited to these samples and extends to microbial populations found in other contexts including culture systems and biofilms.

Culture-independent methods to determine the composition and structure of the the microbiota are described in the art (Pace, Science 276:734-740, 1997). In large part, these methods rely on the retrieval of small subunit, or 16S, rRNA gene sequences, which provide a phylogenetic context in which to describe the diversity of the community. Several methods useful in examining the 16S rRNA-encoding gene are described in Forney et al. (Curr. Opin. Microbiol. 7:210-220, 2004). One method is to directly amplify DNA extracted from a community (a population of microbes) using primers that target the conserved regions of the 16S rRNA-encoding gene. These PCR amplified products are then cloned. Following 16S rRNA-encoding gene clone library construction, the sequences of clones may be determined by conventional sequencing methods.

A method for determining the composition and structure of the microbiota is to determine terminal restriction fragment length polymorphism, or T-RFLP, (Marsh, Curr. Opin. Microbiol. 2:323-327, 1999). T-RFLP targeting 16S rRNA-encoding gene (Avaniss-Aghajani et al., BioTechniques. 17:144-146, 148-149, 1994; E. Avaniss-Aghajani et al., J. Clin. Microbiol. 34:98-102, 1996; Kuehl et al., Infect. And Immun. 73:6952, 2005) has been used to profile complex microbial communities (Bruce and Hughes, Mol. Biotechnol. 16:261-269, 2000; Clement et al., J. Microbiol. Methods. 31:135-142, 1998; W. T. Liu et al. Appl. Environ. Microbiol. 63:4516-4522, 1997).

Preferred methods for performing T-RFLP is described in Blackwood et al. (2003. Appl. Environ. Microbiol. 69:926-932) and Kuehl et al. (2005. Infect. and Immun. 73:6952). In brief, PCR amplification is performed using primers targeting conserved regions of bacterial 16S rRNA genes. One of these primers is linked to a fluorescent dye 6-FAM and the other primer is unlabeled. PCR amplification is performed and the product is purified. The purified PCR product is cut with restriction enzymes and the digest separated on an automated sequence analyzer suitable it fluorescence sequencing (such as the ABI 3100 Genetic Analyzer in GeneScan mode, Applied Biosystems Instruments, Foster City, Calif.). The 5′-terminal restriction fragments (TRFs) are detected by excitation of the 6-FAM molecule attached to one of the primers. The sizes and abundance of the fragments are calculated by GeneScan 3.7.

Profiles can be analyzed using a series of data manipulation and statistical calculations. Calculations are performed on profiles generated by digestion of fluorescently labeled PCR products that have been restriction digested with a single enzyme. A statistical method for determining “true” peaks and comparing electropherograms can be performed as detailed by Abdo et al. (Environ. Microbiol. 8:929-938, 2006). This method employs a purl script that recursively analyzes each electropherogram to distinguish true peaks from background noise and then compares electropherograms and constructs “bins” that contain corresponding TRFs from each electropherogram.

For each electropherogram, the number and height of peaks in each profile is considered to represent the number and relative abundance of different phylotypes present in the sample. Phylotype richness (S) is calculated as the total number of distinct TRF peaks in each normalized profile. The Shannon-Weiner diversity index is described in C. J. Kuehl et al. (Infect. and Immun. 73:6952, 2005) using the statistical program JMP and is herein incorporated by reference.

For construction of 16S rRNA-encoding gene clone library, a preferred method is described in C. J. Kuehl et al. (Infect. and Immun. 73:6952, 2005) and is herein incorporated by reference. Unlabeled primers with sequences identical to those used in T-RFLP analysis are used to amplify DNA samples using the same conditions as those fir T-RFLP analysis. Following purification, PCR products may be cloned and sequenced using conventional molecular biology techniques. Statistical differences in the compositions of clone libraries from experimental and control samples may be determined using LIBSHUFF (version 1.2) (Singleton et al., Appl. Environ. Microbiol. 67:4374-4376, 2001).

### F. Regulatory T Cells

Regulatory T cells can induce peripheral tolerance and limit mucosal reactivity (McGuirk and Mills, Trends Immunol 2002:23(9):450-5). In various animal models, several regulatory T cell phenotypes have been reported. Regulatory T cells express various markers and have been indicated to be involved in different diseases (See, e.g., Field et al., J. Immunol. 170:2508-2515; von Herrath and Harrison Nat Rev Immunol. 3(3):223-32, 2003; Bach, Nat Rev Immunol. 3(3):189-98, 2003; Curotto de Lafaille Curr Opin Immunol. 14(6):771-8, 2003; McGuirk and Mills, Trends Immunol. 23(9):450-5, 2002; Tung et al., Immunol Rev. 82:135-48, 2001; Read and, Powrie, Curr. Opin. Immunol. 13(6):644-649, 2001; Yssel et al., Microbes Infect. 3(11):899-904, 2001). In some systems, they are distinguished through differential expression of surface molecules like CD25 (Shevach, Nature Reviews Immunology 2:389-400, 2002), CD45RB (Annacker and Powrie, Microbes & Infection 4:567-574, 2002), and CTLA-4 (Read et al., J. Exp. Med. 192:295-302, 2000). This pattern of cell surface protein expression suggests that they may be in a primed effector or memory state. These regulatory cells may mediate some of their affects through production of IL10 and TGFβ. Described is an anergic regulatory T cell (Tr1) that produces high levels of IL10 and TFGβ. Another cell called Th3 suppresses induction of experimental autoimmune encephalomyelitis primarily through production of TGFβ. Still others are not dependent on soluble IL10 or TGFβ, but instead express on their surface latency-associated peptide, which is the amino-terminal domain of the TGFβ precursor peptide (Oida et al., J. Immunol. 170(5):2516-22, 2003). Internal cell markers have also been reported for regulatory T cells (Schubert et al., J. Biol. Chem. 276(40):37672-37679, 2001).

1. Determination of Th1 and Th2 Responses

In order to correlate a microbiota profile to an immune response, the Th1 and Th2 response may be distinguished. Metawali et al. (J. Immunol. 157:4546, 1996) has shown that in mice, it is possible to distinguish a Th1 from a Th2 response by histologic analysis, and by analysis of cytokine and immunoglobulin profiles. Further, Sander et al. (J. Exp. Med. 171:2171, 1990) has shown that cell surface expression of Feg3 and MHC Class II molecules afford discrimination. In this procedure, small bowel and colon are examined histologically to determine the degree of mucosal inflammation, eosinophilia and mastocytosis. The latter cell types are indicative of a Th2 response. Mesenteric lymph nodes (MLN) and spleens can be dissociated into single cell suspensions for in vitro culture in microwell plates. Cells (1−2×10⁶/well) in complete RPMI medium are cultured for up to 72 h in the presence or absence of worm antigen or anti-CD3 and then the supernatants are assayed for cytokines and immunoglobulins. IFN-γ, TNFα and IgG2a characterize a Th1 response, whereas IL-4, IL-5, IgE and IgG1 typify a Th2 reaction. Also, serum can be assayed for cytokine and immunoglobulin concentrations. Furthermore, dispersed inflammatory leukocytes are examined by flow cytometry for Feγ3 expression on macrophages (Th1) and MHC Class II expression on B cells (Th2). Controls include serum, MLN and spleens from appropriate age-matched, littermate mice that hosted no parasite.

A similar analysis can differentiate a human Th1 from a Th2 response. One examines inflamed tissue, isolated leukocytes from regions of inflammation and peripheral blood cells. Leukocytes are cultured in vitro alone or in the presence of parasite antigen or antigens to stimulate cytokine release. IgG2 substitutes for IgG2a.

2. Determination of Regulatory T Cell Activity

Methods of isolating regulatory T cells from in vitro culture or animals are known in the art, for example, as described in McGuirk and Mills, Trends Immunol. 23(9):450-455, 2002; Field et al., J. Immunol. 170:2508-2515, 2003; von Herrath and Harrison, Nat. Rev. Immunol., 3(3):223-232, 2003; Francois Bach, J. Nat Rev Immunol., 3(3):189-198, 2003; Curotto de Lafaille and Lafaille, Curr. Opin. Immunol., 14(6):771-8, 2002; McGuirk et al., Trends Immunol. 23(9):450-455, 2002; Tung et al., Immunol. Rev. 182;135-148, 2001; Read and Powrie, Curr. Opin. Immunol. 13(6);644-649, 2001; Yssel et al., J. Microbes Infect. 3(11):899-904, 2001; Shevach, Nature Reviews Immunology 2:389-400, 2002; Annacker and Powrie, Microbes & Infection 4:567-574, 2002; Read et al., J. Exp. Med. 192:295-302, 2000.

Regulatory T response or activity may be measured by an internal marker, a cell surface marker, or a secreted marker as described herein. Useful internal markers for regulatory T cells include, but are not limited to, transcription factors such as Scurfin, Smad7, Gata3, Tbet (Tbx21). Useful surface markers for regulatory T cells include, but are not limited to, CD4, CD45RB^{lo}, CD45Rc, Cytolytic T lymphocyte associated antigen 4 (CTLA-4), CD25, CD103, Ox40, 4-1BB, CD62L, α_{E}β integrin, latency-associated peptide (LAP) or glucocorticoid induced TNF receptor family related protein (GITR), chemokine receptor CCR5, TI-ST2. Useful secreted markers for regulatory T cells include, but are not limited to, IL-5, IL-10 and TGFβ.

3. Non-Limiting Examples of Methods for In-Vitro Measuring the Amount of Markers

Cytokine Detection by Flow Cytometry: Splenocytes, MLN or intestinal inflammatory cells to RPM1 complete medium are placed into 24-well tissue culture plates at 2×10⁶ cells/well. Cells are incubated 4-6 h in the presence or absence of anti-CD3 or appropriate antigen with brefeldin A at 10 μg/ml. Brefeldin prevents exocytosis of proteins and promotes accumulation of the cytokine within the cell. For cytoplasmic cytokine detection, the cells are fixed in 2% paraformaldehyde at room temperature for 5 min following surface staining to distinguish cell subtypes. Cells are washed and re-suspended in 50 μL PBS 0.2% Saponin and 1 μg anti-cytokine antibody and incubated at room temperature for 20 minutes. Next, the cells are washed twice in Saponin and re-suspended in PBS/FCS. The specificity of the cytokine antibody staining is confirmed by pre-blocking the cells with an excess of un-conjugated antibody of the same isotype and cytokine specificity or by incubating the cells in the presence of recombinant cytokine. Phycoerythrin (PE)-labeled irrelevant antibody controls also are included to assess background staining. The cells are analyzed using flow cytometry.

ELISAs: ELISAs measure cytokine and antibody concentrations in cell supernatants from cells cultured in microtiter plates and manipulated as describe above. Many of these assays are already operational within our laboratory. Cytokines are assayed using two monoclonal antibodies (mAbs) in a two-site sandwich ELISA. The anti-cytokine mAbs are purified by ammonium precipitation from supernatants of antibody secreting hybridoma clones. Microtiter plates are coated with 50 μl of 1 μg/ml coating antibody in PBS containing Tween 20 (PBS-T), and incubated at 4° C. overnight. Then, wells are blocked by the addition of 150 μl of 10% FCS in PBS with incubation at 37° C. for 30 minutes. Standards comprise recombinant cytokine or cytokine-containing supernatants from mitogen or antigen-activated spleen cells from schistosomiasis-infected mice. Sample and standard dilutions are made in RPMI containing 10% FCS (complete RPMI) in a separate 96-well flat bottom microtiter plate, and 50 μl volumes are transferred to the ELISA plates that have been washed three times in PBS-T. Samples are incubated in the assay plates for 1 h at 37° C. Appropriate mAb is conjugated to biotin. After washing three times PBS-T, each well will receive 50 μl of antibody-biotin conjugate at 0.5 μg/ml in 1% BSA/PBS-T. Plates are incubated at room temperature for 1 h followed by washing three times in PBS-T. Streptavidin-horseradish peroxidase conjugate (75 μl) is added at 1 μg/ml in 1% BSA/PBS-T and incubated at room temperature for 1 h. Plates are washed 10 times in fresh PBS-T, and 100 μl of substrate (2,2′-azino(3-ethylbenzthiazoline sulfonic acid) at 1 mg/ml in 44 mM Na₂HPO₄, 28 mM citric acid, and 0.003% H₂O₂ is added. The colored product is measured at a wavelength of 405 nm with a reference wavelength of 490 nm, using a multiscan microplate reader.

Immunoglobulins are quantified using anti-isotype specific ELISAs. Affinity-purified goat anti-IgM, -IgG1, -IgG2a, -IgG2b, -IgG3, -IgA and -IgE are used as capture antibodies and absorbed to flexible polyvinyl microtiter dishes at 10 μg/ml. After addition of culture supernatants, incubation and washing, appropriate isotype alkaline-phosphatase-cougated goat anti-Immunoglobulin is used to detect total mouse Immunoglobulin bound to the plates. Standard curves are generated using purified Immunoglobulin. To measure parasite antigen-specific antibody, soluble antigen is biotinylated and used to detect bound mouse Immunoglobulin. The plates are analyzed on to ELISA reader at 410 nm, and concentrations of total Immunoglobulin are determined using the standard curve and best-fit analysis software. Antigen-specific antibody concentrations are compared relative to the O.D. readings since soluble parasite antigen is not a defined antigen that permits precise quantitation.

ELISPOT Assays: ELISPOT assays are established to count lymphocytes secreting either polyelonal antibody or cytokines. Ninety-six-well nitrocellulose-backed microtiter plates are coated overnight at 4° C. with 1 μg/ml of either anti-Immunoglobulin o anti-cytokine antibodies in PBS-T. The plates then are blocked with PBS containing 10% PCS and washed extensively with PBS-T.

Serial dilutions of a single cell suspension, starting with 5×10⁴ cells/well, are incubated on the plate for 5 h at 37° C. in a humidified 5% CO₂ atmosphere. The plates are washed with PBS-T and overlaid with biotinylated anti-Immunoglobulin or -cytokine antibodies overnight at 4° C. Next, plates are washed and treated with streptavidin-glucose oxidase-conjugate for 2 h and washed again.

The antibody or cytokine secreted by single cells is visualized with substrate. The colorimetric reaction is halted after 30 min by washing and spots enumerated under 30× magnification. The dilution of cells producing 10-50 spots/well is used to calculate the total number of secreting cells per sample. Controls include wells coated with inappropriate goat antibody or inappropriate antigen, or left uncoated.

A modification of the assay using soluble antigen to coat the wells permits quantitation of parasite antigen-specific, immunoglobulin secreting B cells also. Briefly, for example, plates are coated with adult T. Maris antigen at 0.25 μg/well or appropriate irrelevant control antigen. Cells are added to the wells after washing. After appropriate incubation, the plates are washed again and treated as described above.

### G. Conditions Treatable According to the Invention

Conditions Related to Altered Microbiota Profile Obesity

Obesity is a clinical condition in which adipose tissue or fatty tissue is increased, in an individual, to a point where the individual is at increased risk for mortality and certain other conditions such as cardiovascular diseases, diabetes mellitus type 2, sleep apnea, and osteoarthritis. In addition, in a non-clinical context, overweight and obese individuals face a heavy social stigma which may take psychological tolls. Overweight and obese children are often shunned by peers. Overweight and obese adults may experience greater difficulty in career advancement.

Obesity, especially central obesity (also known as male-type or waist-predominant obesity), is a risk factor for the “metabolic syndrome,” a clustering of a number of diseases and risk factors that heavily predispose an individual towards cardiovascular disease. People with the metabolic syndrome are at increased risk of coronary heart disease and other diseases related to plaque buildups in artery walls (e.g., stroke and peripheral vascular disease) and type 2 diabetes. The metabolic syndrome has become increasingly common in the United States.

Obesity is also correlated with the following conditions: cardiovascular conditions—congestive heart failure, enlarged heart and associated arrhythmias and dizziness, cor pulmonale, varicose veins, pulmonary embolism; endocrine conditions—polycystic ovarian syndrome (PCOS), menstrual disorders, infertility; gastrointestinal conditions—gastroesophageal reflux disease (GERD), fatty liver disease, cholelithiasis (gallstones), hernia, and colorectal cancer; renal and genitourinary conditions—urinary incontinence, glomerulopathy hypogonadism (in males), breast cancer, uterine cancer, stillbirth; conditions effecting the skin and appendages—stretch marks, acanthosis nigricans, lymphedema, cellulitis, carbuncles, intertrigo; musculoskeletal conditions—hyperuricemia, immobility, osteoarthritis, lower back pain; neurologic conditions—stroke, meralgia paresthetica, headache, carpal tunnel syndrome, dementia; respiratory conditions—dyspnea, obstructive sleep apnea, hypoventilation syndrome, Pickwickian syndrome, asthma; psychological conditions—depression, low self esteem, body dysmorphic disorder, social stigmatization.

Transplantation of gut microbiota from normal mice to germ-free recipients increases their body fat without any increase in food consumption (Backhed et al., Proc. Natl. Acad. Sci. USA 101:15718-15723, 2004). This may be due to a role for microbiota in digestive processes that effect nutrient availability. For example, gut microbiota break down foods that the host cannot digest (Gill et al., Science 312:1355-1359, 2006). Obesity has been correlated to gut microbiota in mice and humans (Ley et al., Proc. Natl. Acad. Sci. USA 102:11070-11075, 2005; Ley et al., Nature 444:1022-1023, 2006). The microbiota of genetically obese ob/ob mice have been shown to be more effective at releasing calories from food during digestion than are the wild-type mice, resulting in increased adiposity (Turnbaugh et al., Nature 444:1027-1031, 2006). A role for microbiota in digestive processes may contribute to obesity. Additionally, obesity has been shown to be linked to inflammation (Stienstra et al., PPAR Res. Nov. 23, 2006). Thus, the effects of gut microbiota on immune responses may also contribute to obesity. Together, these observations suggest that the manipulation of gut microbiota may be a viable approach for the treatment of obesity.

Irritable Bowel Syndrome

Irritable Bowel Syndrome, or spastic colon, is a bowel disorder characterized by bowel dysfunction. The most common symptoms are lower abdominal pain accompanied by disrupted bowel habits and relief upon defecation. Irritable Bowel Disorder may be characterized as diarrhea predominant (IBS-D), constipation-predominant (IBS-C) or with alternating stool pattern (IBS-A). Acute onset IBS may develop following infectious illness and is characterized by two or more of fever, vomiting, acute diarrhea and positive stool culture. Inflammation of gastrointestinal tissues is one aspect of a clinical condition called post-infections IBS (or IBS-PI). It is estimated that up to about 10% of chronic IBS begins with some type of an acute intestinal infection/inflammation (Sangwon et al. J. Gastroenterol. 20:381, 2005). Evidence that gut microbiota contribute to IBS is described by E. Malinen et al. (Am. J. Gastroenterol. ISSN 0002-9270), Fanigliulo et al. (Acta Biomed. 77:85-89, 2006) and Riordan and Kim (Curr. Opin. Gastroenterol. 22:669-73, 7006).

### H. Evaluation of Disease Pathology

The following procedures are utilized to measure the clinical parameters of the noted conditions.

Evaluation of Obesity and Risk for Obesity

In practice, for most examples of overweight that may indicate risk, both clinician and patient determines “by eye” whether excess fat is a concern. Clinical measures are describe henceforth.

Clinically, obesity is measured by Body Mass index (BMI), waist circumference, and evaluating the presence of risk factors or comorbidities (U.S. Dept. of Health and Human Services, National Institutes of Health, “The Practical Guide: Identification, Evaluation and Treatment of Overweight and Obesity in Adults 5” 2000). BMI, or Body Mass Index, is calculated by dividing the subject's weight in kilograms by the square of his/her height in meters. The current definitions set by the World Health Organization (published 2000) are as fellows: a BMI less than 18.5 is underweight, a BMI of 18.5-24.9 is normal weight, a BMI of 25.0-29.9 is overweight; a BMI of 30.0-39.9 is obese, a BMI of 40.0 or higher is severely or morbidly obese, a BMI of 35.0 or higher accompanied by at least one other significant comorbidity is morbidly obese.

An alternate way to determine obesity is to assess percent body fat. In general, men with more than 25% body fat and women with more than 30% body fat, are considered obese. Underwater weighing is generally accepted as a most accurate method for determining body fat. Alternate methods include the skinfold test and bioelectrical impedance analysis. Other methods for measuring body fat include computed tomography, magnetic resonance imaging, and dual energy X-ray absorptiometry.

Waist circumference is also used as an indicator of obesity. In particular, central obesity, which may be measure by waist circumference, has a stronger correlation to cardiovascular disease than BMI. Metrics for waist circumference include but are not limited to absolute waist circumference and waist-hip ratio. An absolute waist circumference greater than 102 cm in men and greater than 88 cm in women or a waist-hip ratio of greather than 0.9 for men and greater than 0.85 for women are used as indicators of central obesity (S. Yusuf et al., Lancet 364:937-952, 2004).

The presence of risk factors and diseases associated with obesity are also used for clinical diagnoses. Life-threatening risk factors include coronary heart disease, type 2 diabetes and sleep apnea. Other risk factors are smoking, hypertension, age and family history.

A combination of genetic and environmental factors may predispose an individual towards becoming overweight or obese. Factors that may indicate that an individual is at risk of becoming obese include but are not limited to genetic factors and some genetic disorders (e.g. Prader-Witti syndrome), underlying illness (hypothyroidism), eating disorders (e.g. binge eating disorder), certain medications (e.g. atypical antipsychotics), sedentary lifestyle, a high glycemic diet, weight cycling, stressful mentality, insufficient sleep, and smoking cessation.

Various genetic factors that predispose an individual towards obesity have been identified such as Prader-Willi syndrome and leptin receptor mutations. Much obesity is likely the result of interactions between multiple genes and non-genetic factors. Certain populations and individuals may be more prone to obesity than others.

Evaluation of IBS

The main symptom of IBS is abdominal pain or discomfort associated with changes in bowel habits in the absence of any apparent structural abnormality. Diagnosis of IBS may utilize the following exemplary criteria but are not limited to them. The Manning Criteria, which distinguishes symptoms arising from other causes in determining a diagnosis, are described in Manning et al. (Br. Med. J. 2:653-4, 1978). The Rome II criteria is described in W. G. Thompson et al., (2000, Functional Bowel Disorder. In: Drossman D A, Corazziari E, Talley N J et al. (eds.), Rome II: The Functional Gastrointestinal Disorders, Diagnosis, Pathophysiology and Treatment, A Multinational Consensus. Lawrence, K S: Allen Press. ISBN 0-9656837-2-9). In addition, IBS is described as diarrhea-predominant (IBS-D), constipation-predominant (IBS-C) or IBS with alternating stool pattern (IBS-A).

In addition to these criteria, patients may have laboratory testing with a complete blood count, basic chemistry panel, and an erythrocyte sedimentation rate. IBS may be diagnosed when Rome II criteria are met, patient history and physical examination to not suggest any other cause, and initial laboratory testing rules out other causes.

Prior to a diagnosis, the following tests may be used to eliminate the possibility that bowel dysfunction is due to other causes: sigmoidoscopy or colonoscopy, esophagogastroduodenoscopy (EGD, gastroscopy), abdominal ultrasound or CT scan, blood tests, assays of stool chemistry (e.g. tests for exocrine pancreas insufficiency), assays for fecal fat, tests for lactose intolerance and fructose malabsorption, deep duodenal biopsy far celiac disease.

### I. Animal Models

Exemplary experimental models useful according to the invention are described in the following references and are herein incorporated by reference. Mouse strains used to study obesity, such as C57BI/6, may be purchased from the Jackson laboratory (Bar Harbor, Me.). Additional non-limiting examples may be found in the art. For example, models of obesity can be found in: Flowers et al., Am. J. Physiol. Endocrinol. Metab. 292:E936-45, 2007; Arsov et al., Biochem. Biophys. Res. Common. 342:1152-9, 2006; Bazhan et al., Ross Fiziol Zh Im I M Sechenova 91:1445-53, 2905; Kiehl et al., Biochem. Biophys. Res. Common. 339:17-24, 2005; Clee et al., Am. J. Ther. 12:491-8, 2005; Alexander et al., Int. J. Obes. 30:50-9, 2006; Jarvis et al., J. Lipid Res. 46:1692-702, 2005; Kim et al., Physiol. Genomics 22:171-81, 2005; Kokkotou et al., Am. J. Physiol. Regul. Integr. Comp. Physiol. 289:R117-24, 2005; Carroll et al., Clin. Dermatol. 22:345-9, 2004; Diament et al., Obes. Rev. 4:249-55, 2003; Svenson et al., J. Appl. Physiol. 94:1650-9, 2093; Anderson, Ann. Med. 28:5-7, 1996; Dong et al., Proc. Natl. Acad. Sci. USA 94:7526-30, 1997; Stubdal et al., Mol. Cell Biol. 20:878-82, 2000; and Faggioni et al., Proc. Natl. Acad. Sci. USA 97:2367-72, 2000. For example, models of irritable bowel syndrome can be found in: Mitch et al., J. Med. Chem. 40:538-46, 1997; Sanger et al., Neurogastroenterol Motil. 10:271-9, 1998; and Martin et al., J. Proteome Res. 5:2185-2193, 2006.

### EXAMPLES

The invention is illustrated by the following nonlimiting examples wherein the following materials and methods are employed.

### Example 1

General Methods

Animals: Colonies of 129/SV IL-10^{-/-} mutant mice, and appropriate control animals are housed in facilities maintained as a specific pathogen-free environment according to standard methods.

Parasite Maintenance, Animal Infection, Production of Schistosome Eggs: The maintenance of T. muris and the method used for infection are as described by Else and Wakelin, 1990, Parasitology, vol. 100, part 3: 479.

Sehistosome eggs were harvested from the livers of schistosome-infected hamsters and stored as described by Elliott, 1996, Methods: A Companion to Methods in Enzymology, 9:255. Five infected hamsters yield about 2×10⁶ eggs.

Preparation of T. suis Eggs: The following process can be used in the preparation and harvesting of T. suis eggs. Adult T. suis worms are isolated from the colon of pigs 7-8 wks after exposure to an experimental inoculation of T. suis eggs. Embryonated eggs are obtained by culturing adult worms hi vitro, and then the excreted eggs, separated from the culture medium by centrifugation, are placed into 0.2% potassium dichromate solution at 22° C. fro 5-6 wks with bubbling to obtain infective first-stage larvae. Eggs are washed twice in sterile water by centrifugation at 1200.times.g for 10 min, counted, and re-suspended in the desired amount of saline based on a calculated dose of 2,500. These eggs are stored for use in the subjects. The ova are stable for at least one year in the refrigerator. To assure infectivity, patients are monitored for the appearance of ova in the stool after colonization. The number of ova in the stool is proportional to the intensity of toe infestation. Also, from tune to tune, pigs are infected with stored ova to assure continued infectivity.

IL-10^{-/-} knockout mouse strain: IL-10 is an important immunoregulatory cytokine that down modulates macrophage activation and accessory cell function (Moore et al., 1993, Ann. Rev. Immunol., 11: 165). Mice rendered IL-10 deficient by targeted gene disruption (IL-10^{-/-}) develop intestinal inflammation (Kuhn et al., Cell 75:263, 1993). The intestinal inflammation is attenuated by treatment with anti-IFNγ antibody demonstrating that inflammation results from overly exuberant Th1 responses to colonic contents (Berg, et al., Invest. 98:1010, 1996). IL-10^{-/-} mice on the 129 and C57B1/6 backgrounds were used.

Infection with M. avium: Mice were infected by injecting 10⁶ colony forming units (CFU) of Mycobacterium avium (ATCC 25291) intraperitoneally. On day 60 of infection, some mice also received 35 S. mansoni cercariae to induce dual infection.

Schistosome Infection and Isolation of Ova: Some experiments used mice (18-20 g) infected for 8-9 wks S. mansoni. Mice were infected subcutaneously with 40 cercariae from the Puerto Rican strain.

Mice were colonized with H. polygyrus by gastric gavage. Controls were sham-treated.

Evaluation of Mucosal Inflammation: To grade intestinal inflammation, tissue may be removed, Swiss-rolled and embedded in paraffin according to standard methods. The sections are stained with hematoxylin and eosin. The degree of colonic inflammation is graded semiquantitatively from 0 to 4 in a blinded fashion by a single pathologist using standardization techniques known in the art.

Methods of obtaining tissue samples are known in the art. Given as examples, samples may be obtained by biopsy or necropsy (Swidsinski et al., Gastroenterology 122:44-54, 2002; C. J. Kuehl et al., Infect. and Immun. 73:6952-6961, 2005).

Methods for extracting microbial populations from tissue samples are known in the art (C. J. Kuehl et al., Infection and Immunity 73:6952-6961, 2006). For example, a method for obtaining samples from the human colon are described in Swidsinski et al. (Gastroenterology 122:44-54, 2002). The method, described in brief, is as follows. Biopsies (3.2-3.6 mm³) are taken during colonoscopy with standard forceps from the ileum and ascending and sigmoid colon and processed immediately (biopsy washing and the setting of cultures) in the laboratory next to the endoscopy room. Each biopsy is first washed in 500 μL of physiologic saline with 0.016% dithioerythritol to remove the mucus, then washed 3 times in 500 μL of physiologic saline by shaking for 30 seconds each time. The supernatants from the second and fourth wash are used to evaluate the superficial microbiota. After the fourth wash, the biopsy is hypotonically lysed by vortexing for 30 minutes in distilled water. The debris left after the hypotonic lysis is evaluated for mucosal bacteria. Methods such as this may be modified for other tissue samples.

### 2. Example 2

Th2 Response to S. mansoni Down-Modulates an Ongoing Th1 Response to an Unrelated Bacterial Th1-Inducing Antigen

It is well established that Th cell immune responses can polarize into Th1 or Th2 patterns. This polarization occurs because IFNγ from Th1 cells inhibits proliferation of Th2 cells, while IL-4 and IL-10 from Th2 cells inhibits development of Th1 cells. The following experiments demonstrated that schistosomiasis alters the murine Th1 response to an established mycobacterial infection.

Mice were co-infected with M. avium and S. mansoni to evaluate the host response to these distinctly different Th1 and Th2 inflammatory stimuli. BALB/cAnN mice develop chronic M. avium infection when injected with this organism (10⁶ CFU). Sixty days after establishment of the mycobacterial infection, the mice were infected with S. mansoni (40 cercariae). The mice were killed sixty days later. Control groups included mice receiving either M. avium only for 120 days or S. mansoni only for 60 days. Dispersed splenocytes or isolated granuloma cells from these animals were cultured in vitro (4×10⁵ cells/welt) for 48 h in the presence or absence of schistosome egg antigen (SEA, a strong Th2 antigen) or mycobacterial antigens purified protein derivative (PPD, a strong Th1-inducing antigen) used at optimal concentration. After the incubation, supernatants were assayed for cytokine or immunoglobulin production using ELISAS. The data in FIGS. 1-3 are mean values, +-SD of three separate experiments.

Splenocytes from mice infected only with M. avium secreted large amounts of IFNγ following stimulation with PPD (Th1 antigen). Spleen cells from uninfected control mice produced none. Most importantly, no IFNγ was detected in spleen cell cultures from concurrently infected mice (M. avium alone vs. concurrent infection, P<0.001, FIG. 1). Soluble schistosome egg antigen (SEA, Th2 antigen) stimulated only IL-4 and IL-5 release from splenocytes of S. mansoni-infected animals. Mice singularly infected with M. avium produced no IL-4 or IL-5 in response to PPD or SEA. However, splenocytes from co-infected animals secreted some IL-4 following PPD stimulation (FIG. 1).

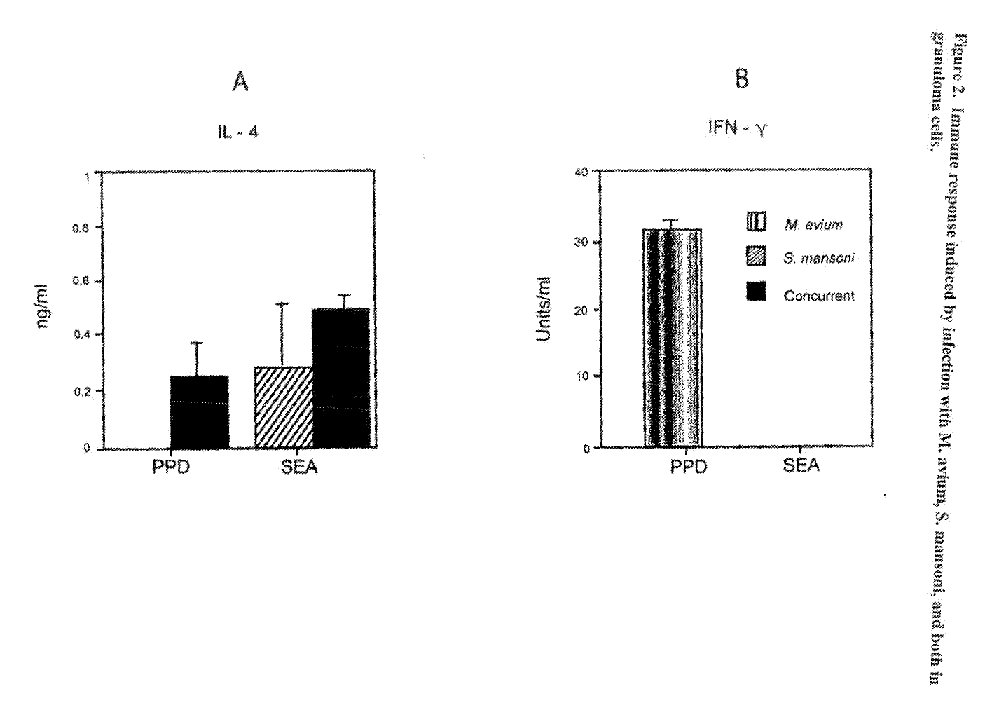

Granulomas were isolated from the livers of mice infected with M. avium or S. mansoni, or from animals that had concurrent infection. Concurrently infected animals develop liver granulomas that contain both schistosome eggs and mycobacteria readily evident on histological examination. Dispersed granuloma cells from these animals were cultured in vitro for 48 h in the presence or absence of SEA or PPD used at optimal concentration. Granuloma cells from mice only infected with M. avium secreted large amounts of IFNγ following stimulation with PPD. No IFNγ was detected in granuloma cell cultures from concurrently infected mice (M. avium alone vs other, P<0.001) (FIG. 2). SEA stimulated IL-4 release from granuloma cells of S. mansoni-animals. SEA did not promote IFNγ secretion under any circumstance. Mice singularly infected with M. avium produced no IL-4 in response to PPD. However, granuloma cells from co-infected animals secreted some IL-4 following PPD stimulation FIG. 2).

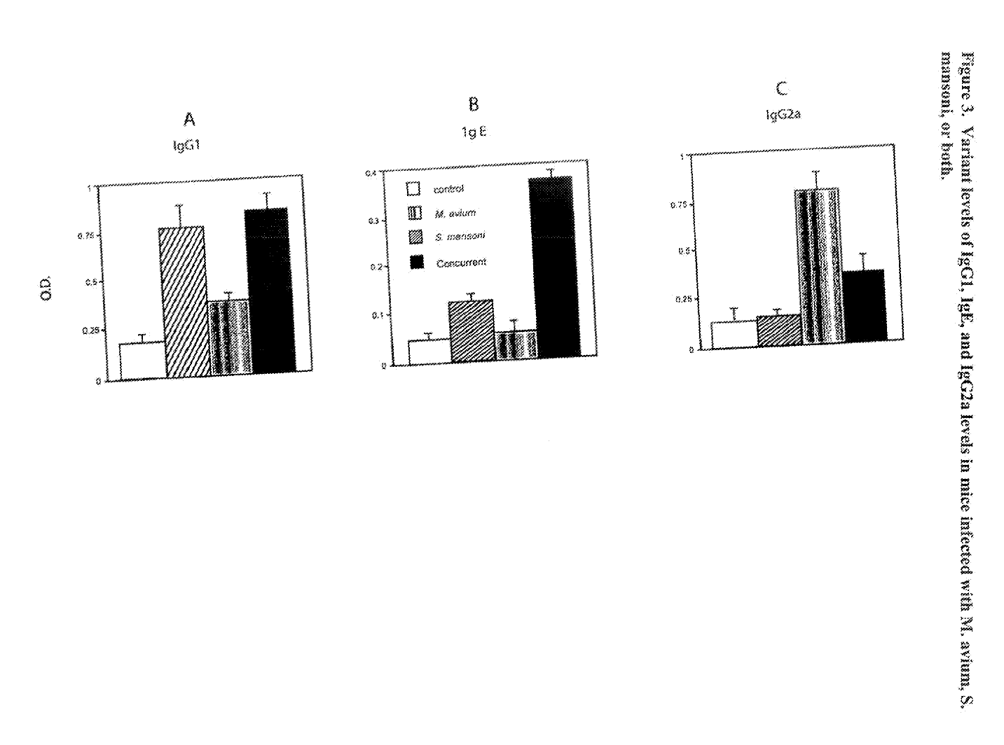

Th1 responses promote IgG2a production, whereas Th2 reactions enhance IgG1 and IgE. FIG. 3 shows that mice infected with M. avium have high serum IgG2a levels. Yet, co-infected animals have normal serum IgG2a concentrations, but increased IgG1 and IgE levels. These data taken together show that a Th2 response to a helminthic infection can down-modulate the ongoing host response to even a strong Th1-inducing organism like M. avium.

### 3. Example 3

C57BL/6 IL-10^{-/-} and wildtype C75BL/6 mice were infected with H. polygyrus. DNA were extracted from the terminal ileum, cecum, proximal and distan colon of animals 2 weeks after challenge with H. polygyrus ova. A combination of 165 rRNA-encoding gene clone library analysis and 16S-based terminal restriction fragment length polymorphism (T-RFLP) analysis were used to compare the structure of the various microbiota employing standard ecologic measures of community diversity.

T-RFLP analysis all H. polygyrus infection was associated with reproducible alterations in the community structure of the mucosa-associated microbiota of the distal gastrointestinal tract. 16S clone library analysis of mucosa from infected and uninfected wild-type mice confirmed that specific perturbations of the mucosa-associated bacterial community followed the introduction of H. polygyrus. 16S clone library analysis was able to identity specific groups of bacteria that changed in relative abundance following helminth infection. Comparison between wild-type and IL-10−/− mice with H. polygyrus results in significant shifts in the microbial community of the distal gastrointestinal tract. As H. polygyrus colonizes the duodenum, these changes may be due to an indirect effect of the alteration of mucosal cytokine profiles due to H. polygyrus infection. This was also suggested by the baseline differences between wild-type and IL-10−/− mice. Alternately, H. polygyrus may elaborate soluble factors into the GI tract lumen that can influence the downstream microbes. One possibility is that specific changes in GI microbiota play a direct role in the amelioration of IBD by H. polygyrus by altering the microbial antigens that serve to drive the proinflammatory cytokine response.

### OTHER EMBODIMENTS

The foregoing examples demonstrate experiments performed and contemplated by the present inventors in making and carrying nut the invention. It is believed that these examples include a disclosure of techniques which serve to both apprise the art of the practice of the invention and to demonstrate its usefulness. it will be appreciated by those of skill in the art that the techniques and embodiments disclosed herein are preferred embodiments only that in general numerous equivalent methods and techniques may be employed to achieve the same result.

A number of embodiments of the invention have been described. Nevertheless, it will be understood that various modifications may be made without departing from the spirit and scope of the invention. Accordingly, other embodiments are within the scope of the following claims.

1. A method for treating an individual who is obese or at risk of becoming obese, the method comprising administering to the individual a physiologically acceptable composition comprising a helminthic parasite, or an active variant thereof, that is of a type and present in an amount sufficient to treat the individual for obesity or reduce the risk of obesity.

2. A method for treating an individual who has Irritable Bowel Syndrome (IBS), the method comprising administering to the individual a physiologically acceptable composition comprising a helminthic parasite, or an active variant thereof, that is of a type and present in an amount sufficient to treat the IBS.

3. The method of claim 1, wherein the helminthic parasite is one that naturally colonizes humans.

4. The method of claim 3, wherein the helminthic parasite is a nematode or platyhelminth.

5. The method of claim 4, wherein the helminthic parasite is a nematode of the genus Ancylostroma, Ascaris, Enterobius, Hymenolepis, Necator, Nippostrongylus, Strongyloides, Trichinella, or Trichuris.

6. The method of claim 5, wherein the helminthic parasite is Ancylostoma duodenale, Ancylostoma brasiliense, Ascaris lumbricoides, Enterobius vermicularis, Necator americanus, Strongyloides stercoralis, or Trichuris trichiura.

7. The method of claim 4, wherein the platyhelminth is a trematode or cestode.

8. The method of claim 4, wherein the platyhelminth resides predominantly in the human intestine.

9. The method of claim 4, wherein the platyhelminth is of the genus Fasciolopsis, Echinostoma, or Heterophyes.

10. The method of claim 1, wherein the helminthic parasite is one that naturally colonizes an animal other than a human.

11. The method of claim 10, wherein the animal other than a human is a mouse, rat, pig, dog, cat, marine mammal or bird.

12. The method of 10, wherein the helminthic parasite can also colonize humans.

13. The method of claim 10, wherein the helminthic parasite is of the genus Angiostrongylus, Heligmosomoides, Nippostrongylus, or Trichuris.

14. The method of claim 13, wherein the helminthic parasite is Heligmosomoides polygyrus, Nippostrongylus brasiliensis, or Trichuris muris.

15. The method of any of claim 10, wherein the helminthic parasite is of the genus Ancylostoma, Anisakis, Ascaris, Gnathstoma, Hymenolepis, Pseudoterranova, Trichinella, Trichuris, or Toxocara.

16. The method of claim 10, wherein the helminthic parasite is Ascaris suum, Hymenolepis nana, Trichinella spiralis, Trichuris vulpi, or Trichuris suis.

17. The method of claim 10, wherein the helminthic parasite is S. douthitti, Trichobilharzia ocellato, T. stagnicolae, T. physellae, or Gigantobilharzia huronensis.

18. The method of claim 1, wherein the helminthic parasite is Clonorchis sinensis, Opisthorchis viverrini, Opisthorchis felineus, Fasciola hepatica, or a parasite of the genus Schistosoma.

19. The method of claim 1, wherein the helminthic parasite is Schistosoma mansoni.

20. The method of claim 1, wherein the helminthic parasite is a parasite of the Diphyllobothrium species, Taenia saginata, or Taenia solium.

21. The method of claim 1, wherein the helminthic parasite is a filarial parasite or a lung fluke.

22. The method of claim 1, wherein the active variant comprises an extract of the helminthic parasite; an ovum or ovum extract of the helminthic parasite; an egg or egg extract of the helminthic parasite; a larvae or larval extract of the helminthic parasite; or a cercariae or cercariae extract of the helminthic parasite.

23. The method of claim 1, wherein the active variant comprises an isolated component of the helminthic parasite.

24. The method of claim 23, wherein the isolated component is an expression product of helminthic parasite genomic DNA sequence.

25. The method of claim 1, wherein the composition comprises a combination of helminthic parasite active variants.

26. The method of claim 1, wherein the helminthic parasite or the active variant thereof alters the microbiota profile of the individual.

27. The method of claim 1, wherein the individual is a human.

28. The method of claim 1, wherein the method further comprises a step of identifying an individual in need of treatment.

29. The method of claim 1, wherein the composition is formulated for oral administration.

30. The method of claim 29, wherein the composition further comprises a filler, carrier, or excipient.

31. The method of claim 1, wherein the composition comprises about 50 to about 50,000 helminthic parasites.

32. The method of claim 1, wherein administering the composition is repeated at least twice.

33. The method of claim 32, wherein administering the composition is repeated approximately weekly or approximately monthly.

34. A method of identifying a helminthic parasite composition useful in the method of claim 1, the method comprising (a) providing a helminthic parasite composition; (b) providing a population of microbes of determined microbiota profile; (c) exposing the population of microbes to the helminth parasite composition, and (d) determining the microbiota profile of the population of microbes, wherein a helminth parasite composition that alters the microbiota profile of the population is identified.

35. The method of claim 34, wherein the microbiota profile is altered to include a greater number or proportion of Bacteroidetes and a lesser number or proportion of Firmicutes.

36. The method of claim 34, wherein the population of microbes is a population present in vivo.

37. The method of claim 36, wherein an immune response is assayed.

38. The method of claim 34, wherein the population of microbes is a population present ex vivo.

39. The method of claim 34, wherein the population present ex vivo is a population maintained in cell culture.
